(19) United States
(12) Patent Application Publication
(10) Pub. No.: US 2013/0302253 A1
(43) Pub. Date: Nov. 14, 2013

(54) CARRIERS FOR THE LOCAL RELEASE OF HYDROPHILIC PRODRUGS

(71) Applicants: Holger Gruell, Eindhoven (NL); Sander Langereis, Mierlo (NL); Charles Sio, Eindhoven (NL)

(72) Inventors: Holger Gruell, Eindhoven (NL); Sander Langereis, Mierlo (NL); Charles Sio, Eindhoven (NL)

(73) Assignee: KONINKLIJKE PHILIPS N.V., EINDHOVEN (NL)

(21) Appl. No.: 13/981,438

(22) PCT Filed: Jan. 25, 2012
(86) PCT No.: PCT/IB12/50347

§ 371 Date: Jul. 24, 2013

(30) Foreign Application Priority Data
Jan. 28, 2011 (EP) 11152529.1

(51) Int. Cl. A61K 9/127; A61K 9/00

(52) U.S. Cl. CPC A61K 9/127; A61K 9/0087
USPC 424/9.37; 424/489; 424/450; 514/254.11

(57) ABSTRACT

Disclosed is a carrier for the local, targeted administration of a hydrophobic drug. The hydrophobic drug is rendered in to a hydrophilic prodrug thereof, and is contained in the lumen of a thermosensitive liposome or polymersome. Upon administration of the carrier, heat can be applied at the locus where the drug is to be released. After release of the prodrug, it will be activated so as to turn into the active drug.

### FIELD OF THE INVENTION

The invention relates the targeted, local delivery of hydrophobic drugs via the release, from a carrier, of a hydrophilic prodrug thereof. The invention also relates to a novel use of thermosensitive carriers.

### BACKGROUND OF THE INVENTION

Many diseases that are mostly localized in a certain tissue are treated with systemically administered drugs. A well-known example of standard cancer therapy is a systemic chemotherapy coming along with significant side effects for the patient due to undesired biodistribution and toxicity. The therapeutic window of these drugs is usually defined by the minimal required therapeutic concentration in the diseased tissue on the one hand, and the toxic effects in non-targeted organs, e.g. liver, spleen, on the other. Localized treatment by, for example, local release of cytostatics from nanocarriers promises a more efficient treatment and a larger therapeutic window compared to standard therapeutics. Localized drug delivery is also important if other therapeutic options such as surgery are too risky as is often the case for liver cancers. Localized drug delivery can also become the preferred treatment option for many indications in cardiovascular disease (CVD), such as atherosclerosis in the coronary arteries.

A promising technology for the localized delivery of drugs, is by administering them via carriers such as liposomes. Liposomes are generally characterized by a lipid bilayer enclosing a cavity. Such a bilayer generally comprises amphiphilic molecules, having the lipophilic moieties of either layer oriented towards each other, and as a result having hydrophilic moieties oriented towards the outside of the liposome as well as towards the enclosed cavity. As a result, the inside of the liposome (i.e. the cavity) is normally aqueous.

This set-up presents a challenge in the event that hydrophobic drugs are to be administered. An example of a hydrophobic anti-cancer drug is docetaxel. Such drugs are difficult, if not altogether impossible, to encapsulate and retain in the cavity (lumen) of liposomes.

Zhigaltsev et al., Journal of Controlled. Release, J. Control.Release (2010), doi:10.1016/j.jconre1.2010.02.029, addresses this by presenting a hydrophilic prodrug of docetaxel and incorporating this into the lumen (cavity) of a non-temperature sensitive liposome. It is reported that such a hydrophilized prodrug can be efficiently retained in a liposomal nanoparticle (LNP), and that release rates can be regulated by varying the lipid composition of the LNP carrier.

The foregoing presents a problem for practical application, as the requirements for being retained (while in circulation) and for being released (when at a desired locus) are almost irreconcilable. Moreover, since the encapsulated substance is necessarily a prodrug, and its action is intended to be local, the delivery will desirably go with measures to secure that the prodrug is not transformed into an active drug until it is at the right spot and on the right time. This is essentially different from prodrugs that are administered systemically, and which circulate (and e.g. can be metabolized) prior to exerting their action.

A further issue is that the above-mentioned existing solution to cast a balance between retaining and release, by varying the lipid composition, detracts from the usefulness of the concept for true targeted delivery, as even from subject to subject the release rates may be different and, obviously, the composition cannot be adapted on an individual basis.

It would be desired to provide a drug delivery system by which hydrophobic drugs can be delivered and activated locally. Particularly, it would be desired to provide such a system that would work reliably in a number of different subjects, notably without having to change the composition of the carrier.

### SUMMARY OF THE INVENTION

In order to better address the aforementioned desires, in one aspect, the invention presents a pharmaceutical composition for the localized delivery of a hydrophobic drug, said composition comprising a thermosensitive carrier comprising a shell enclosing a cavity, and wherein said substance contained in the cavity is a hydrophilic prodrug of the hydrophobic drug.

In another aspect, the invention is the use of a thermosensitive carrier for the administration of a hydrophilic prodrug of a hydrophobic drug.

In a further aspect, a method is presented for the local administration of a hydrophobic drug, said method comprising administering a carrier comprising a hydrophilic prodrug of the hydrophobic drug, the carrier being a thermosensitive liposome.

### BRIEF DESCRIPTION OF THE DRAWINGS

FIG. 1 presents a schematic drawing of the triggered release and in situ activation of a hydrophilic prodrug (indicate by the connected colored circles and squares) from the lumen of a thermosensitive liposome;

FIG. 2 presents a schematic drawing of the triggered release and in situ activation of a hydrophilic prodrug from the lumen of a thermosensitive liposome, together with a co-encapsulated MRI contrast agent;

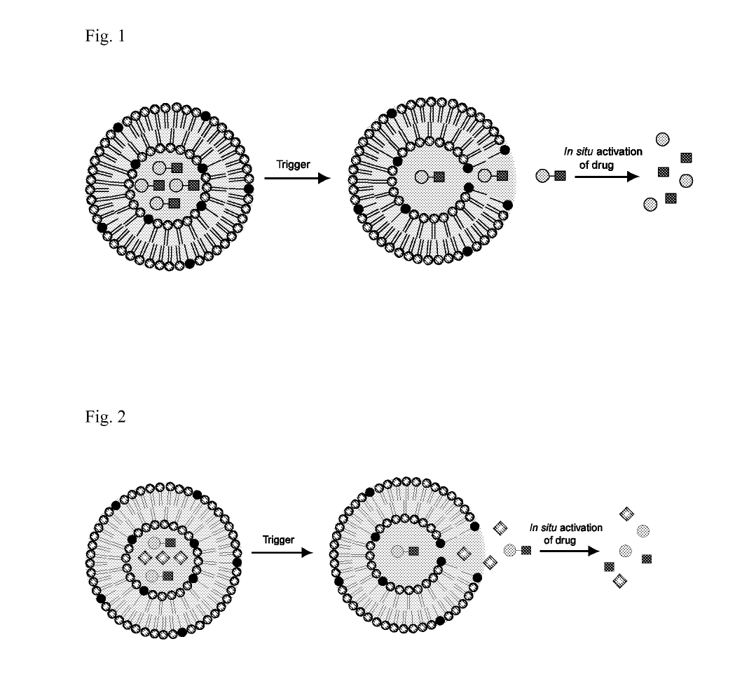

### DETAILED DESCRIPTION OF THE INVENTION

In a broad sense, the invention can be described with reference to the judicious insight that thermosensitive liposomes are capable of solving several technical problems associated with the local delivery of hydrophobic drugs. It will be understood that this concept can equivalently also be applied to a broader area than only thermosensitive liposomes, viz. in fact to any other carriers (particularly nanocarriers such polymersomes or liposomes) that are capable of releasing their contents as a result of a local stimulus.

The release, through a local stimulus, of specifically a hydrophilic prodrug contained in the cavity of a carrier, such as the lumen of a liposome, provides the possibility for a timed release of the prodrug. This, in turn, carries the potential advantage that the activation of the prodrug into the active form of the drug can be carried through at the time when the prodrug is released.

The present invention will further be described with respect to particular embodiments and with reference to certain drawings but the invention is not limited thereto but only by the claims. Any reference signs in the claims shall not be construed as limiting the scope. The drawings described are only schematic and are non-limiting. In the drawings, the size of some of the elements may be exaggerated and not drawn on scale for illustrative purposes. Where the term “comprising” is used in the present description and claims, it does not exclude other elements or steps. Where an indefinite or definite article is used when referring to a singular noun e.g. “a” or “an”, “the”, this includes a plural of that noun unless something else is specifically stated.

The invention employs carriers that are thermosensitive. This means that the physical or chemical state of the carrier is dependent on its temperature.

It will be appreciated by the skilled person that the thermosensitive nature of a carrier should be understood in the context of administration to animal subjects, preferably human subjects. I.e., the temperatures at which a change will occur in the carrier so as to release it contents (e.g. by opening up the lipid bilayer of a thermosensitive liposome) are generally within a level that can be tolerated by a subject, i.e. normally below 50° C., and preferably 1-5 degrees above body-temperature.

Thermosensitive carriers for use in the invention ideally retain their structure at about 37° C., i.e. human body temperature, but are destroyed at a higher temperature, preferably only slightly elevated above human body temperature, and preferably also above pyrexic body temperature. Typically about 42° C. (mild hyperthermia) is a highly useful temperature for thermally induced (local) drug delivery. Heat can be applied in any physiologically acceptable way, preferably by using a focused energy source capable of inducing highly localized hyperthermia. The energy can be provided through, e.g., microwaves, ultrasound, magnetic induction, infrared or light energy.

Carriers of the invention include but are not limited to thermosensitive micro- and nanoparticles, thermosensitive polymersomes, thermosensitive nanovesicles and thermosensitive nanospheres, all based on polymers.

Thermosensitive nanovesicles generally have a diameter of up to 100 nm. In the context of this invention, vesicles larger than 100 nm, typically up to 5000 nm, are considered as microvesicles. The word vesicle describes any type of micro- or nanovesicle.

Preferred carriers comprise a shell that encloses a cavity, such as liposomes or polymersomes, wherein the shell's integrity can be affected by the external influence of heat.

Thermosensitive liposomes include but are not limited to any liposome, including those having a prolonged half-life, e.g. PEGylated liposomes. Thermosensitive liposomes for use in the invention ideally retain their structure at about 37° , i.e. human body temperature, but are destroyed at a higher temperature, preferably only slightly elevated above human body temperature, and preferably also above pyrexic body temperature. Typically about 42° C. is a highly useful temperature for thermally guided drug delivery. The required heat to raise the temperature of the thermosensitive drug carriers so as to promote the destruction of the thermosensitive carriers may be used. Heat can be applied in any physiologically acceptable way, preferably by using a focused energy source capable of inducing highly localized hyperthermia.

The energy can be provided through, e.g., microwaves, ultrasound, magnetic induction, infrared or light energy. Thermosensitive liposomes are known in the art. Liposomes according to the present invention may be prepared by any of a variety of techniques that are known in the art. See, e.g., U.S. Pat. No. 4,235,871; Published PCT applications WO 96/14057; New RRC, Liposomes: A practical approach, IRL Press, Oxford (1990), pages 33-104; Lasic,D.D., Liposomes from physics to applications, Elsevier Science Publishers, Amsterdam, 1993; Liposomes, Marcel Dekker, Inc., New York (1983). See also WO 2009/059449 for preferred thermosensitive liposomes that can be used in the present invention.

Preferred liposomes comprise both a short and a long chain, as explained below. This refers to any phospho lipids that can be incorporated into the lipid bilayer of a liposome, and which essentially comprise a short and a long alkyl chain are present.

The lipid bilayer in these mixed short/long chain liposomes preferably comprises a phospholipid having two terminal alkyl chains, one being a short chain having a chain length of at most fourteen carbon atoms, the other being a long chain having a chain length of at least fifteen carbon atoms.

Conceivably, the long alkyl chain comprises a double bond, but saturated chains are preferred. According to the invention, the lengths of these chains can be varied in order to tune the lipid bilayer properties.

It will be understood that the terms “short” and “long” in their most general sense are relative. I.e., if the short chain has two carbon atoms, a chain having more than six carbon atoms could be considered long. On the other hand, if the long chain has fifteen carbon atoms, a chain having ten carbon atoms could be considered short. In general, the difference in length between the short chain and the long chain will be at least two carbon atoms, preferably at least eight carbon atoms, and most preferably between eleven and sixteen carbon atoms.

The short chain preferably has a length of length of at most fourteen carbon atoms, more preferably at most ten carbon atoms, and most preferably at most five carbon atoms. In preferred embodiments, the short chain has a length of two, three, four, or five carbon atoms. The long chain preferably has a chain length of at least ten carbon atoms, more preferably at least fifteen carbon atoms. The upper limit for the long chain preferably is thirty carbon atoms, more preferably twenty carbon atoms. In preferred embodiments the long chain has fifteen, sixteen, seventeen, or eighteen carbon atoms.

Phospho lipids are known and generally refer to phosphatidylcho line, phosphatidyl-ethanolamine, phosphatidylserine and phosphatidylinositol. In the invention it is preferred to employ phosphatidylcholine.

In a further preferred embodiment, the mixed short chain/long chain phospholipids satisfy either of the following formula (I) or (II).

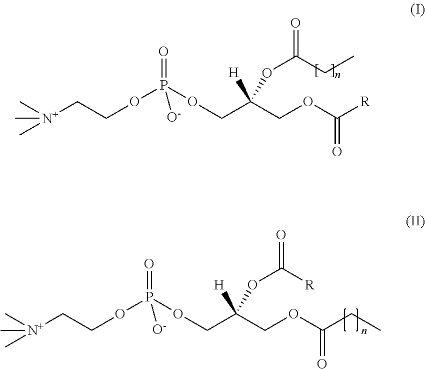

Herein R is an alkyl chain of fifteen to thirty carbon atoms, and is preferably C₁₅H₃₁ or C₁₇H₃₅; n is an integer of 1 to 10, preferably 1 to 4.

These compounds can be synthesized by esterification of lyso-PC with the corresponding anhydrides. An exemplified reaction scheme is given in Scheme 1 below:

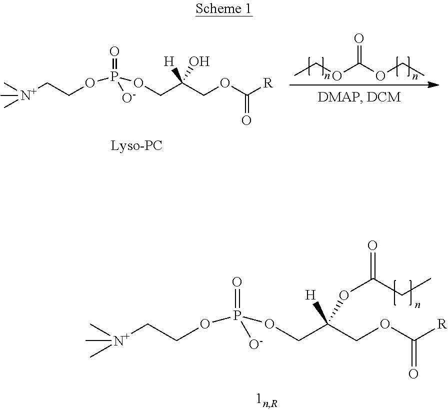

Herein DMAP stands for 4-dimethyl amino pyridine and DCM stands for dichloro methane. The indication 1_{n,R} refers to the compound of formula (I) above.

Other preferred thermosensitive liposomes for use in the present invention are those described by Lindner et al. in Journal of Controlled Release 125 (2008), 112-120. These liposomes are based on hexadecylposphocholine (miltefosine). Still other preferred thermosensitive liposomes are those containing MPPC (1-Myristoyl,2-Palmitoyl-sn-Glycero 3-PhosphoCholine) and MSPC (1-myristoyl-2-stearoylphosphatidylcholine).

Different approaches have been used to produce thermosensitive liposomes for controlled release, such as using the phase transition property of the constituent lipids [G. R. Anyarambhatla, D. Needham, Enhancement of the phase transition permeability of DPPC liposomes by incorporation of MPPC: a new temperature-sensitive liposome for use with mild hyperthermia, Journal of Liposome Research 9(4) (1999) 491-506]. For example, dipalmitoyl-phosphatidylcholine (DPPC) having a phase transition temperature of 42.5° C. is the most notable lipid. In order to reduce the drug leakage from these liposomes, cholesterol is commonly added as a lipid component. The addition of cholesterol reduces the thermal sensitivity of DPPC in cholesterol-containing liposomes. This technique has met with various degrees of success [G. R. Anyarambhatla, D. Needham, Enhancement of the phase transition permeability of DPPC liposomes by incorporation of MPPC: a new temperature-sensitive liposome for use with mild hyperthermia, Journal of Liposome Research 9(4) (1999) 491-506; M. H. Gaber, K. Hong, S. K. Huang, D. Papahadjoupoulos, Thermosensitive sterically stabilized liposomes: formulation and in vitro studies on mechanisms of doxorubicin release by bovine serum and human plasma. Pharm. Res. 12 (1995) 1407-16].

Thermosensitive liposomes have been known to have the capability of encapsulating drugs and releasing these drugs into heated tissue. Recently, successful targeted chemotherapy delivery to brain tumors in animals using thermosensitive liposomes has been demonstrated [K. Kakinuma et al, “Drug delivery to the brain using thermosensitive liposome and local hyperthermia”, International J. of Hyperthermia, Vol. 12, No. 1, pp. 157-165, 1996]. Kakinuma's study was conducted by using an invasive needle hyperthermia RF antenna placed directly within the tumor to locally heat the tumor and the liposomes. The results showed that when thermosensitive liposomes are used as the drug carrier, significant drug levels were measured within brain tumors that were heated to the range of about 41-44° C. A minimal invasive targeted treatment of large tumor is also disclosed in U.S. Pat. No. 5,810,888 Entrapment of a drug or other bio-active agent within liposomes of the present invention may also be carried out using any conventional method in the art. In preparing liposome compositions of the present invention, stabilizers such as antioxidants and other additives may be used as long as they do not interfere with the purpose of the invention. Examples include co-polymers of N-isopropylacrylamide (Bioconjug. Chem. 10:412-8 (1999)).

In use, thermosensitive liposomes are delivered to a subject and a target area in the subject is heated. When the thermosensitive liposome reaches the heated area, it undergoes a gel to liquid phase transition and releases the active agent. The success of this technique requires a liposome with a gel to liquid phase transition temperature within the range of temperatures that are obtainable in the subject.

The foregoing holds, mutatis mutandis, for thermosensitive polymersomes. Thermosensitive polymersomes include those having a prolonged half-life, e.g. PEGylated polymersomes. The term “polymersomes” is used here to generally indicate nanovesicles or microvesicles comprising a polymeric shell that encloses a cavity. These vesicles are preferably composed of block copolymer amphiphiles. These synthetic amphiphiles have an amphiphilicity similar to that of lipids. By virtue of their amphiphilic nature (having a more hydrophilic head and a more hydrophobic tail), the block copolymers will self-assemble into a head-to-tail and tail-to-head bilayer structure similar to that of liposomes.

Compared to liposomes, polymersomes have much larger molecular weights, with number average molecular weights typically ranging from 1000 to 100,000, preferably of from 2500 to 50,000 and more preferably of from 5000 to 25000.

References on environment-sensitive carriers are e.g. U.S. Pat. No. 6,726,925, US 2006/0057192, US 2007/0077230A1 and JP 2006-306794. Further reference is particularly made to Ahmed, F.; Discher, D. E. Journal of Controlled Release 2004, 96, (1), 37-53; to Ahmed, F.; Pakunlu, R. I.; Srinivas, G.; Brannan, A.; Bates, F.; Klein, M. L.; Minko, T.; Discher, D. E.

Molecular Pharmaceutics 2006, 3, (3), 340-350; and to Ghoroghchian, P. P.; Frail, P. R.; Susumu, K.; Blessington, D.; Brannan, A. K.; Bates, F. S.; Chance, B.; Hammer, D. A.; Therien, M. J. Proceedings of the National Academy of Sciences of the United States of America 2005, 102, (8), 2922-2927.

Entrapment of a drug or other bio-active agent within carriers of the present invention can be carried out using any conventional method in the art.

Thermosensitive liposomes of the invention can be administered to a subject using any suitable route, for example, intravenous administration, intra-arterial administration, intramuscular administration, intraperitoneal administration, subcutaneous, intradermal intraarticular, intrathecal intracerebroventricular, nasal spray, pulmonary inhalation, oral administration as well as other suitable routes of administration known to those skilled in the art. Tissues which can be treating using the methods of the present invention include, but are not limited to, nasal, pulmonary, liver, kidney, bone, soft tissue, muscle, adrenal tissue and breast. Tissues that can be treated include both cancerous tissue, otherwise diseased or compromised tissue, as well as healthy tissue if so desired. Any tissue or bodily fluid that can be heated to a temperature above 39.5 ° C. may be treated with the liposomes of the invention.

The dose of active agent may be adjusted as is known in the art depending upon the active agent comprised in the carrier.

The target tissue of the subject may be heated before and/or during and/or after administration of the thermosensitive liposomes of the invention. In one embodiment, the target tissue is heated first (for example, for 10 to 30 minutes) and the liposomes of the invention are delivered into the subject as soon after heating as practicable. In another embodiment, thermosensitive liposomes of the invention are delivered to the subject and the target tissue is heated as soon as practicable after the administration.

Any suitable means of heating the target tissue may be used, for example, application of radio frequency radiation, application of ultrasound which may be high intensity focused ultrasound, application of microwave radiation, any source that generates infrared radiation such as a warm water bath, light, as well as externally or internally applied radiation such as that generated by radioisotopes, electrical and magnetic fields, and/or combinations of the above.

In preparing polymersome compositions of the present invention, stabilizers such as antioxidants and other additives may be used as long as they do not interfere with the purpose of the invention. Examples include co-polymers of N-isopropylacrylamide (Bioconjug. Chem. 10:412-8 (1999)).

In view of the applicability in agents for medical diagnostics and treatment, it is preferred that the polymeric blocks are made of pharmaceutically acceptable polymers. Examples hereof are e.g. polymersomes as disclosed in US 2005/0048110 and polymersomes comprising thermo-responsive block co-polymers as disclosed in WO 2007/075502. Further references to materials for polymersomes include WO 2007081991, WO 2006080849, US 20050003016, US 20050019265, and US-6835394.

The invention is directed to the delivery of hydrophilic prodrugs of hydrophobic drugs. This presents a novel concept for the local temperature-triggered release of hydrophilic prodrugs from the lumen of a temperature-sensitive liposome followed by in situ activation of the drug.

Local temperature increase can be induced by any heat source such as light, radiofrequency, alternating magnetic field in combination with magnetic particles, or ultrasound. The latter preferably is performed under MRI guidance (MRgHIFU), where the MRI allows procedure planning and provides a temperature feedback to the ultrasound. In this setting, the temperature-induced (pro-)drug release can also be monitored by releasing co-encapsulated MR imaging probes for image guided drug release.

For carrying out embodiments in which the local drug delivery from thermosensitive liposomes or polymersomes is combined with magnetic resonance imaging, reference is made to WO 2009/69051 and WO 2009/72079.

The hydrophilic prodrugs refer to any compound that is sufficiently hydrophilic to be retained in the lumen (cavity) of a liposome or polymersome.

An example of a hydrophilic prodrug is docetaxel modified with N-methyl-piperazinyl butanoic acid.

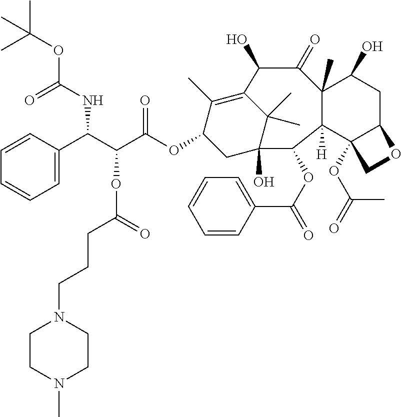

In general, once it has been established that it is desired to provide a hydrophobic prodrug of a hydrophilic drug, the person skilled in the art will be able to modify the hydrophobic drug accordingly. This will generally be by the addition of side chains or substitution groups or other moieties of a hydrophilic nature. It will be understood that such side chains, groups, or moieties will have to allow being removed once the prodrug has entered the subject's system.

The invention is generally applicable to prodrugs that satisfy the following requirements: they are hydrophilic (capable of being retained in the lumen of a liposome during administration and localization); they are capable of being modified into the (hydrophobic) drug itself as a result of the exposure to the local environment where the drug is intended to act.

This local environment can refer, e.g., to pH, or to circulating enzymes that metabolize the prodrug into the active drug and a prosthetic group. These enzymes are, e.g. proteases, which are highly abundant enzymes present everywhere in the body, and to which the prodrug will only be exposed as a result of the local release.

Without this reference intended to be limiting, a preferred group of drugs that can be used in the present invention is disclosed in WO 2009/141738. This document is expressly referred to and, where legally possible incorporated by reference, as an enabling disclosure of suitable prodrugs that can be retained in the lumen of a liposome.

These preferred hydrophilic prodrugs are generally weakly basic derivatives of a drug, provided with a hydrophilic group. The weak alkalinity of the prodrug makes it possible to retain the prodrug, in a stable state, at an acidic pH. Upon release into the physiological environment of a subject, at a physiological pH, the ester bond will be hydrolyzed, and the hydrophobic drug is formed in situ.

The selection of thermosensitive carriers for the foregoing type of weakly alkaline prodrugs brings addresses a further issue. The mechanism of release from the carrier not being by mere diffusion, but by the actual opening up of the carrier, a relatively fast, if not immediate, exchange can take place of the originally slightly acidic environment within the carrier, and the physiological bulk environment surrounding the carrier. In practice this means that the prodrug nearly simultaneously with its release, if not already at the onset of release, will be in the active form.

In connection with the prodrug-loaded thermosensitive carriers of the invention, it can be advantageous to also include, in or on the carrier, one or more contrast agents for magnetic resonance imaging. Thus, in one embodiment, the invention also pertains to a composition as described above, further comprising a magnetic resonance imaging contrast agent selected from the group consisting of ¹⁹F MR contrast agents,. ¹H MR contrast agents, Chemical Exchange-dependent Saturation Transfer (CEST) contrast agent, and combinations thereof. Such agents are known. References regarding the incorporation into liposomes (or other carriers also capable of drug delivery) are, e.g., WO 2009/069051, WO 2009/072079, WO 200/060403.

In a further aspect, a method is presented for the local administration of a hydrophobic drug, said method comprising administering a carrier comprising a hydrophilic prodrug of the hydrophobic drug, the carrier being a thermosensitive liposome.

The method of the invention can be carried out in accordance with a variety of protocols. Examples thereof are the following:

Protocol 1: inject formulation while hyperthermia is maintained as long as possible and reasonable. In this protocol, mainly intervascular release will take place with subsequent diffusion/uptake of the prodrug in the souring tissue.

Protocol 2: inject formulation, wait for extravasation of the liposomal-prodrug particle (e.g. 24 to 48 hours, depending on the biodistribution), then activate the release of the prodrug by applying local temperature increase.

Protocol 3: combine protocol 1 or 2 with a pretreatment, for example hyperthermia, or cavitation to enhance drug uptake into tissue, before applying protocol 1 or 2.

1. A pharmaceutical composition for the localized delivery of a hydrophobic drug, said composition comprising a thermosensitive carrier comprising a shell enclosing a cavity, and wherein the cavity contains a hydrophilic prodrug of the hydrophobic drug.

2. A composition according to claim 1, wherein the carrier is a thermosensitive liposome or polymersome.

3. A composition according to claim 2, wherein the liposome is selected from the group consisting of liposomes comprising hexadecylposphocholine (miltefosine), liposomes comprising 1-myristoyl,2-palmitoyl-sn-glycero 3-pho sphocholine, liposomes comprising 1-myristoyl-2-stearoylphosphatidylcholine, and liposomes the lipid bilayer of which comprises a phospholipid having two terminal alkyl chains, one being a short chain having a chain length of at most fourteen carbon atoms, the other being a long chain having a chain length of at least fifteen carbon atoms.

4. A composition according to any one of the preceding claims, wherein the hydrophilic prodrug is a weakly basic derivative of the drug provided with a hydrophilic group, comprising an ester bond that, at a physiological pH, is hydrolyzed so as to release form of the hydrophobic drug.

5. A composition according to claim 4, wherein the hydrophilic prodrug is docetaxel modified with N-methyl-piperazinyl butanoic acid satisfying the following formula:

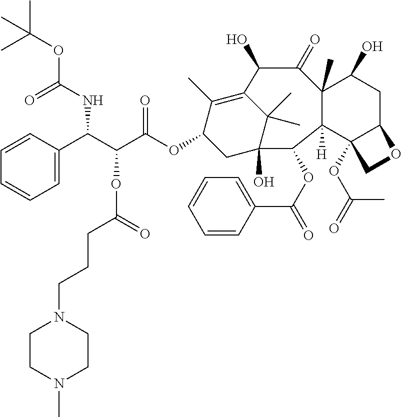

6. A composition according to any one of the preceding claims, further comprising a magnetic resonance imaging contrast agent selected from the group consisting of ¹⁹F MR contrast agents,.¹H MR contrast agents, Chemical Exchange-dependent Saturation Transfer (CEST) contrast agent, and combinations thereof.

7. The use of a thermosensitive carrier for the administration of a hydrophilic prodrug of a hydrophobic drug method comprising the steps of:
(a) providing a hydrophobic drug;
(b) modifying the hydrophobic drug so as to provide a hydrophilic prodrug thereof;
(c) providing a thermosensitive carrier comprising a shell enclosing a cavity;
(d) allowing the hydrophilic prodrug to be contained in the cavity.

8. (canceled)

9. (canceled)

10. (canceled)

11. (canceled)

12. (canceled)

13. (canceled)

14. (canceled)
